(19) United States
(12) Patent Application Publication
(10) Pub. No.: US 2016/0317442 A1
(43) Pub. Date: Nov. 3, 2016

(54) Nanosomal preparation of the complex formed by quercetin (or another flavonol, flavone or a derivative thereof) and 2-hydroxypropyl-beta-cyclodextrin for intravenous use in cerebral pathological conditions

(71) Applicants: Lucia VAAMONDE, (US); Federico DAJAS, (US); Antonio TEDESCO, (US); Fernanda BLASINA, (US)

(72) Inventors: Federico DAJAS, Montevideo (UY); Antonio TEDESCO, Ribeirao Preto, SP (BR); Fernanda BLASINA, Montevideo (UY); Lucia VAAMONDE, Montevideo (UY); Marcela DIAZ, Montevideo (UY)

(21) Appl. No.: 14/433,734

(22) PCT Filed: Oct. 2, 2013
(86) PCT No.: PCT/IB2013/059067

§ 371 Date: Apr. 6, 2015

(51) Int. Cl. A61K 9/127; A61K 47/48; A61K 31/352; A61K 9/00

(52) U.S. Cl. CPC A61K 9/1271; A61K 9/0019; A61K 47/48969; A61K 31/352; A61K 9/1278

(57) ABSTRACT

The invention involves the preparation of cholesterol lecithin nanosomes, without propylene glycol, from the complex formed by quercetin (or another flavonol or flavone or a derivative thereof) and 2-hydroxypropyl-β-cyclodextrin, by means of a process that allows the safe, effective intravenous use thereof in the treatment of cerebral pathological conditions in adults and newborns. The preparation is safe, stabilizes the altered haemodynamic parameters in severe neonatal hypoxia in newborn pigs and is effective in protecting cerebral function in experimental Parkinson's disease models and in newborn pigs subject to hypoxia.

### BACKGROUND TO JUSTIFY THE NEED OF THE INVENTION

### a) Importance of Neurological Diseases in Adults.

The Cerebrovascular Attack (CVA) involves focal or massive neuronal death in the brain, making it one of the leading causes of death and disability in Uruguay and the world. Since about 8% of CVA end in death and the incidence is at about 90/100,000, these diseases represent a huge impact on the community for its high morbidity, its high and the affectation in quality [1, 2].

On the other hand, between 20 and 70% of those who survive a stroke persist with sequelae causing varying degrees of disability.

Taking U.S. figures, there are nearly one million of new stroke cases each year, with a high incidence of mortality (1 in 17 deaths, 12%), being the third cause of death, only behind heart disease and cancer [2 ].

Despite this situation, there is a lack of specific therapies, being plasminogen activators the only therapeutic alternative approved by the Food and Drug Administration (FDA).

### b) Importance and Severity of Neurological Diseases in the Newborn.

Asphyxia of the newborns is an issue of great importance. The World Health Organization found that of a total of about 5 million neonatal deaths worldwide, 28% are caused by asphyxia and trauma. In the period 2000-2003, 20% of neonatal deaths in the Region of the Americas (excluding Canada and the United States) were because of asphyxia at birth. This causes an estimated 33,000 deaths from neonatal asphyxia annually in Latin America.

As a result of asphyxia an hypoxic-ischemic brain damage occurs that leads to chronic neurological disorders and death, with an incidence of 0.5 to 1 per 1000 live births in developed countries [8]. This situation is worse in developing countries because there are more pregnancies without adequate control and more non-institutional deliveries. The brain damage may be moderate with a risk of death of 6%, and up to 30% of survivors have disabling sequelae. When damage is severe, the mortality rate rises to 60% and total survivors show persistent sequelae [9].

### c) Lack of Therapeutic Resources for Acute Cerebral Pathology

The devastating consequences of hypoxia-ischemia in the adult and perinatal brain seem inevitable at present due to the inefficiency of the treatment options available.

Various treatments with neuroprotective potential have been tried. Among them, since the sequence of events triggered by toxic ischemia involves the production of free radicals and reactive oxygen species, substances with antioxidant activity have been tested [13, 14]. The evidence so far does not justify the recommendation of the routine use of any of these agents and hypothermia is the only therapeutic measure for perinatal asphyxia.

From the lack of effective therapies for brain damage arises the current need of characterizing compounds with the potential to provide protection to brain tissue in situations of acute hypoxic-ischemic injury and neurodegenerative disorders.

### d) Search for New Therapeutic Resources

The search for antioxidants is one of the most investigated therapeutic approaches for cerebral hypoxic-ischemic damage. The strategies are related to the synthesis of new compounds or finding and characterization of natural compounds with antioxidant potential. These include flavonoids such as quercetin.

Quercetin is a powerful antioxidant flavonoid, widely distributed in nature where it is found in fruits and vegetables [16].

Much evidence in neurons in culture has shown the protective effect of quercetin versus different oxidative aggressions [17,18]. The in vivo evidences are less numerous and have been mostly observed in experiments with chronic oral administration for more than 7 days (17). Cases of beneficial action after acute administration have been described in trauma or transient cerebral ischemia after intraperitoneal administration. Similar number of studies has shown negative results, especially in experimental models Parkinson's disease [17]. These conflicting results are attributed to the fact that quercetin does not cross the blood-brain barrier while positive experimental situations involved the breaking of this barrier allowing the cerebral passage of quercetin. Furthermore, the channels used experimentally (oral and intraperitoneal) are not suitable for acute conditions of cerebral pathology.

On the other hand, quercetin suffers important hepatic metabolism (glucuronidation, methylation, etc.) due to which the circulating levels of free flavonoid are very low. If this is added to the lipophilic nature of quercetin that difficult dissolution in water-soluble media for access to the brain, the need for a carrier that will protect the molecule of the cerebral metabolism providing access to the brain appears as a key requirement to get in vivo the therapeutic effect observed in neurons in culture.

### BRIEF DESCRIPTION OF THE DRAWINGS

FIG. 1 is an electrocardiogram record, oxygen saturation, systemic blood pressure and pulmonary arterial pressure. The latter increases after administration of liposomes.

FIG. 2 is microphotographs of a phase contrast microscope showing quercetin crystals present in A disappearing in B.

FIG. 3 is a measurement of particle size and stability of liposomes with and without PEG (con PEG, sin PEG, in the tittles, respectively) Zeta Sizer, describing the zeta potential of the liposomes, size (open bars) and dispersion (polydispersity index: linear curve).

FIG. 4 is a photograph taken at 72000× magnification by electron microscopy.

FIG. 5 is physiological records before and after administration of nansomes.

FIG. 6 is a diagram recording systemic blood pressure throughout the hypozia experiment in a newborn, as an example of the variability caused by hypoxia and stability provided by nanosomes.

FIG. 7 is a diagram showing values of the upper limit of the amplitude of the electrical activity in the EEG. Basal, post resuscitation and eight hours post hypoxia (*=p<0.05), d values shown.

FIG. 8 is a diagram showing levels of dopamine in the striatum of rats lesioned in the substantia nigra with the toxin 6—OHDA in an experimental model of Parkinson's disease. Significant decrease after injury (white bar) and statistically significant recovery after intravenous administration 1 h and 24 h after the nanosomal preparation was observed after. Injected 1 h after injury, nanosomes without quercetin, were also able to recover dopamine, but not at 24 h. (HPBCD in the figure).

### DESCRIPTION OF THE INVENTION

### a) Immediate Antecedents

The set of results obtained by the group of innovators in experimental studies in vitro confirmed the antioxidant and neuroprotective role of quercetin. [20-23].

However, in aqueous preparations, quercetin is not detected in the brain due to its low bioavailability.

For the purpose of providing a vehicle to quercetin to facilitate its access to the brain, the inventors formulated in first instance liposomes containing only a mixture of lecithin with quercetin. Cerebral protection was then demonstrated experimentally in a model of ischemia in the rat, when the liposomes were injected intraperitoneally (22).

Liposomes have been widely used in the pharmaceutical industry in a variety of clinical applications to transport and deliver a wide range of active ingredients [50,51].

It should be noted that, for use in conditions such as stroke or asphyxia of the newborn, the preparation of quercetin should be administered acutely by the intravenous route.

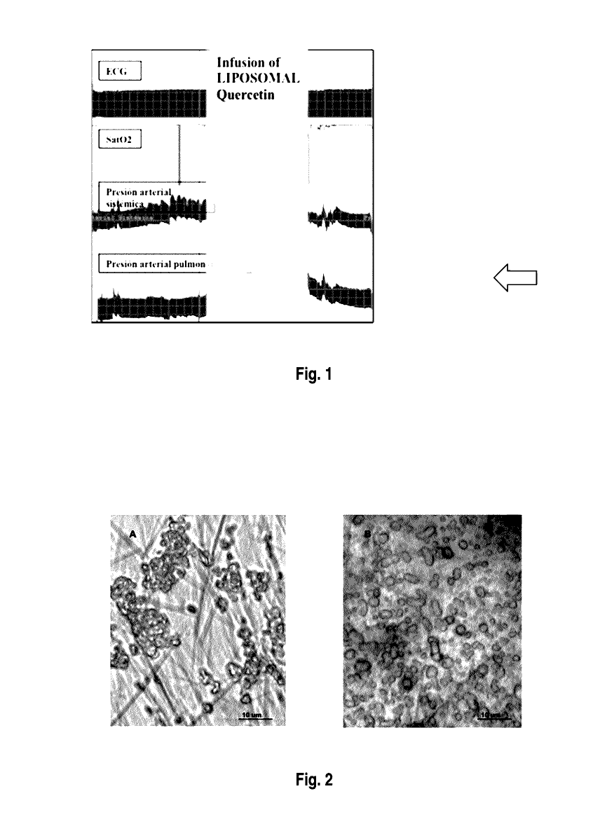

In research conducted by the inventors, they were able to show that the preparation of liposomes of lecithin and quercetin that had been beneficial effects administered intraperitoneally in rats (27), produced significant adverse effects when administered intravenously in pigs. This is evident when the effect on the pressure of the pulmonary arterial is observed in FIG. 1, where an increase during the intravenous administration of a preparation of liposomes with quercetin under baseline conditions is analyzed (arrow).

Since there are no formulations for intravenous use, a formulation of a nanosomal carrier was first obtained with the addition of cholesterol to improve solubility. Quercetin crystals decreased as it can be observed in the in the microphotographs of phase contrast microscope as shown in A and B in FIG. 2, where the crystals present in A, disappear in B.

### b) Advantages of the Invention Over the Prior Art

To improve bioavailability 2-hydroxypropyl-cyclodextrin (HPBCD) was added to the nanosome preparation. In our case the HPBCD is used since it improves solubility and is non-toxic in several animal models and in humans, including children [32]. It has been reported that quercetin complexes with HPBCD resulting in a molecular encapsulation that has even been postulated for cerebral use in previous studies (Chinese patent documents CN101301477B and CN101301477A).

This innovation differs from these previous ones in the fact that beyond obtaining the quercetin/HPBCD complex it was encapsulated in a lecithin/cholesterol nanosome. The novelty regarding the effects on cerebral protection is that the complex quercetin/HPBCD achieves synergistic neuroprotective action, an aspect shown by us in the protection model in Experimental Parkinson and not mentioned in the Chinese patents listed above.

This innovation differs from the state of the art in the field in that it does not use polyethylene glycol (PEG) in its formulation. Quercetin and PEG-liposomes given orally have been studied in mice with solid tumors [28,29] and anxiety in adult rats [1].

In the patent application CN102058536A of May 2011 the combination of phospholipid/cholesterol with cyclodextrins (CDs) and PEG was used in order to obtain greater stability and solubility of liposomes.

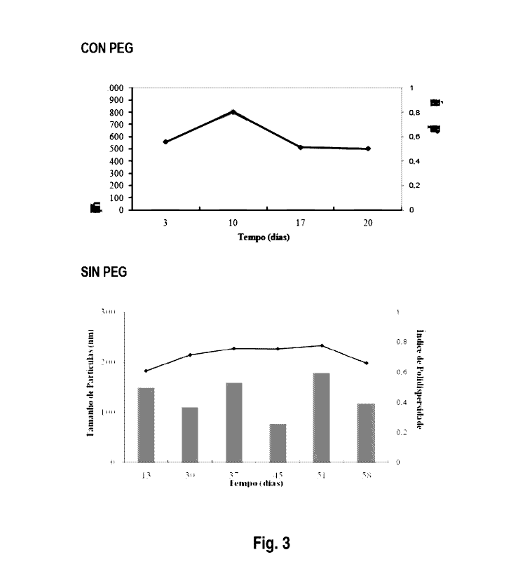

Studies conducted by the inventors including PEG in the nanosomes showed lower size stability of nanosomes as assessed with or without PEG in the preparation, when the size is determined by polydispersity index up to two months after its preparation (FIG. 3).

Furthermore it has been shown that the general use of glycols in solubilizing drugs is associated with adverse effects such as phlebitis, pain and hemolysis. Particularly in the pediatric and neonatal age (under 4 years) and in patients with impaired alcohol deshldrogenase enzyme or low glomerular filtration or liver disease or pregnancy, they are prone to accumulation of glycols increasing therefore the risk of toxicity. In the neonatal period, no risk assessment of PEG has been done and its use is not recommended, for example by ESNEE (European Study for Neonatal Exposure Excipients). Because of these reasons PEG is not included in the formulation proposed in this patent, making a distinct difference with available nanosomal preparations, and making also a difference in reference to obviousness.

Against this background it can be concluded that though liposomal preparations of quercetin have already been used for neuron recovery, the present preparation differs from the state of art in the size (<200 nm) of the particles, in which it does not include PEG in the formulation and in that the active molecule is not only the quercetin molecule, but the quercetin/HPBCD complex. Besides, the resulting preparation has adequate hemodynamic tolerance for intravenous administration.

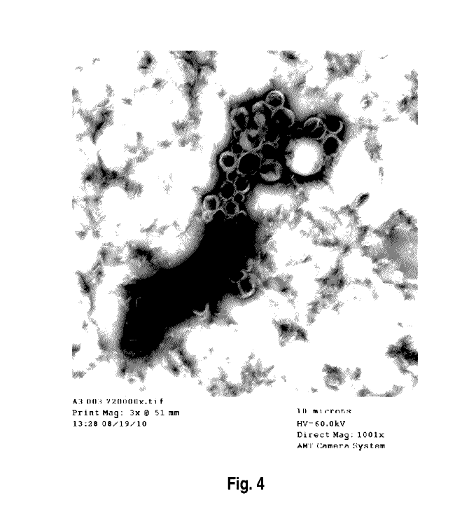

Although it may seem obvious to include HPBCD in a liposome, from the data available it should be noted that the inventors obtained HPBCD experimental evidence showing that it is necessary for the safety of intravenous administration of the formulation (FIG. 4).

The present invention also relates to a method of preparing an injectable formulation for intravenous administration comprising nanosomes of lecithin/cholesterol complex with quercetin or other flavone or flavonol or its alkyl and/or sulfur derivatives with 2-hydroxypropyl- -cyclodextrin, prepared according to the following steps:

I—Preparation of the complex mixing 2-hydroxypropyl-β-cyclodextrin and quercetin or other flavonol or flavone on a ratio of quercetin or other flavonol or flavone to 2-hydroxypropyl-β-cyclodextrin of 1:20 to 1:50 times by a period of time of 12 to 90 hours, in a medium containing ethanol with continuous stirring in sterile conditions.

I—Inclusion of complex obtained in step I into a nanosome of lecithin/cholesterol in a ratio of 5 to 10 respectively, with sonication for a period of 5 min to 2 hours.

III—The preparation obtained in step II is mixed with the complex obtained in step I in a ratio of flavonoid/cholesterol of 0.5 to 2 times.

IV—The formulation obtained in III is injected in a saline solution at a temperature between 60 and 100 degrees Celsius at a rate of 5 to 30 ml/h to result in the formation of nanosomes.

c. Embodiment of the Invention

For the preparation of lipid nanosomes cover cholesterol plus lecithin are used.

For incorporation of quercetin/HPBCD in the bilayer of phosphatidylcholine and cholesterol, 0.5 to 5 ml of ethanol solution are mixed with 30-95% quercetin and HP -β-CD (ratio 1-20 to 50 times).

This solution is maintained in a laminar flow chamber with magnetic stirring over 48 h to form complexes between the components. Subsequently cholesterol and phosphatidylcholine are added to this mixture and saline, pH between 6.2 and 7.8 is injected. The injection is made between 40 and 80° C. in a reactor, with magnetic stirring and with a variable flow.

Throughout the procedure aseptic conditions are kept to allow the production of a preparation that can be used intravenously.

In FIG. 4, an electron microscopy image of the nanosomes can be observed.

### C. Testing

To evaluate the safety and effectiveness of the nanosomal preparation the following tests were performed.

a—Security: Tolerance and Hemodynamics Security in Rats and Newborn Pigs

The preparation obtained according to the previously described formulation was evaluated in its hemodynamic tolerance in rats and newborn pigs. The latter were anesthetized with electrocardiographic monitoring, and monitoring of systemic blood pressure (SBP), pulmonary pressure (PAP), heart rate (HR), core temperature (TC), and oxygen saturation (Sat O₂). Acid-base status, electrolytes, hemoglobin, hematocrit, pH and blood gases were also evaluated.

The nanosomal preparation of quercetin/HPBCD was injected to the animal intravenously at quercetin concentrations of 10 mg/kg.

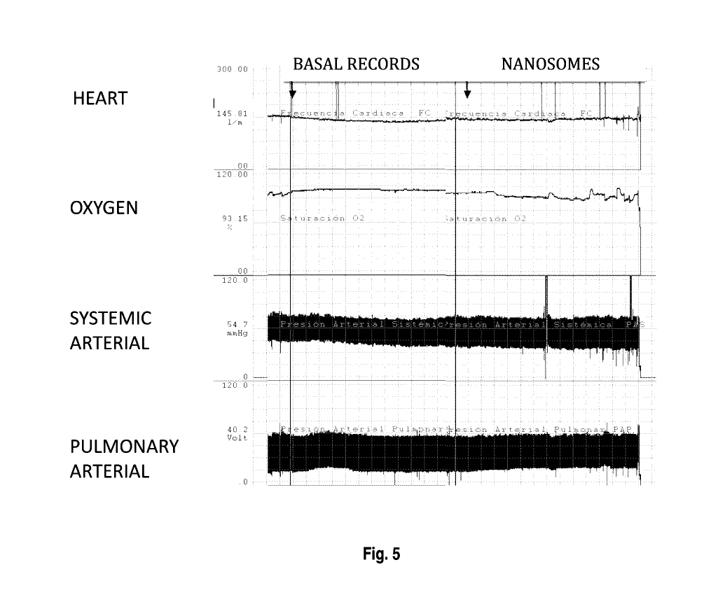

As shown in FIG. 5, no changes were observed in HR, Sat O₂, SBP, and PAP after administration of nanosomes, demonstrating the hemodynamic safety as the basis for the systemic administration of the preparation.

Administered intravenously to rats, via the femoral vein with continuous flow for 30 minutes at the same quercetin concentration of 10 mg/kg, the nanosomal preparation of quercetin/HPBCD showed no clinical changes.

b—Neuroprotective Effectiveness. Stabilization of Hemodynamic Parameters Altered By Severe Hypoxia in Newborn Pigs

### O₂ Saturation and Inspired Oxygen Fraction

In newborn pigs anesthetized and monitored as described previously, hypoxia was induced decreasing inspired oxygen by the endotracheal tube to 8%. During hypoxia, the O₂-saturation level decreases to below 20%.

After completion of the hypoxia the fraction of inspired oxygen (FiO₂) is set to 1 for 30 min, to start resuscitation. After that time it is maintained for 8 h at adequate levels (above 90%) applying an increased fraction of inspired O₂ if it drops below the minimum required.

To get adequate oxygen saturations animals that had been subjected to hypoxia required more oxygen (FiO₂ 0.34±0.25), while those receiving hypoxia and the nanosomal preparation required an FiO₂ of 0.22±0.04, significantly reduced as demonstrated in Table 1, which shows a chi square analysis of the proportion of animals requiring only air or oxygen.

TABLE I
Distribution of the proportion of animals requiring air or oxygen
to maintain saturation >90%.

| | | Supplementary |
|---|---|---|
| | Air | oxygen |
| Control (sham) | 1 | 0 |
| Hypoxia | 0.3 | 0.7 |
| Hypoxia + nanosomes at | 0.77 | 0.22* |
| a Quercetin | | |
| concentration of | | |
| 10 mg/kg | | |

*= P < 0.05 (Chi square/Fischer).

These results indicate a lower requirement for oxygen in the group treated with the nanosomal preparation to maintain an adequate exchange and to deliver to different organs an appropriate oxygen supply. In particular they indicate a better physiological condition, with protection of lung injury induced by the hypoxic event and subsequent re oxygenation.

### Systemic Arterial Pressure

A progressive fall in systemic blood pressure, which often fails to maintain the required levels for life is observed throughout the experiment. After completion of hypoxia several animals showed a tendency to hypotension, making necessary to add inotropic medication to maintain stability. In this case continuous infusion of adrenaline was used according to the requirement of each animal.

This hemodynamic response is more stable in the animals that received the preparation in doses of 10 mg/kg quercetin. Those animals maintained systemic blood pressure levels next to the controls without the need for inotropic addition.

TABLE II
Proportion of animals that required the addition of epinephrine to maintain
stable systemic arterial pressure (*= p < 0.05, square/Fischer Chi).

| | Adrenaline | No Adrenaline |
|---|---|---|
| Control (sham) | 1 | 0 |
| Hypoxia | 0.38 | 0.62 |
| Hypoxia + nanosomes at a | 0 | 1* |
| Quercetin concentration of | | |
| 10 mg/kg | | |

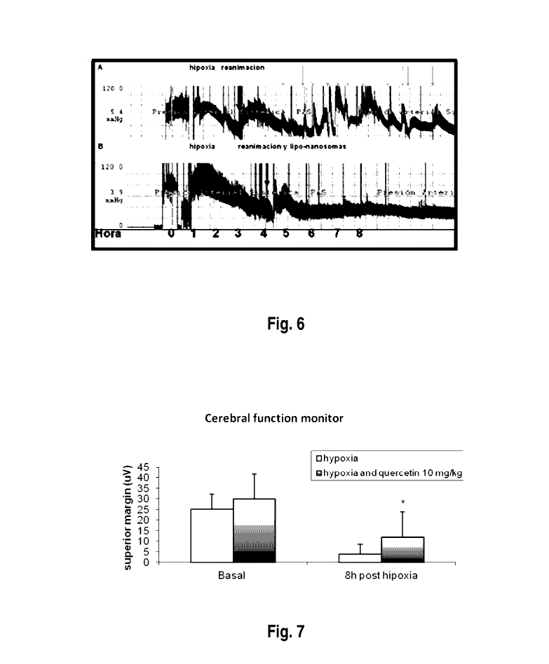

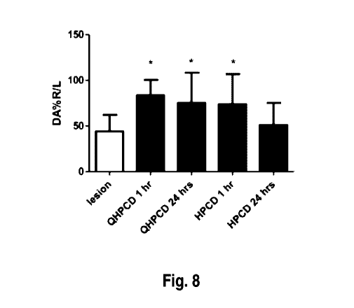

In FIG. 6, the monitoring of SBP is observed. The SBP is stable after injury in animals that received the nanosomal preparation (bottom records), while it was instable subsequent to resuscitation in only hypoxic newborn piglets (up records in figure), with repeated episodes of hypotension, which were only reversed with the addition of adrenaline.

### Acid-base Balance and Metabolism

With respect to acid-base balance, electrolytes and glucose metabolism, no significant differences between the hypoxia group and the group that also received single-dose of 10 mg/kg quercetin were detected.

c. Neuroprotective Effectiveness II: Improvement of Brain Electrical Activity By Treatment with Nanosomes in Newborn Piglets Subjected to Hypoxia.

In newborn pigs subjected to severe hypoxia by restricting the fraction of oxygen received in the same model as previously described, electrical brain activity was recorded with an electroencephalogram amplitude monitor (CFM). During the experimental situation (hypoxia), the amplitude of cerebral activity is maintained below a value of 7 μν for a minimum time of 17 min. During hypoxia amplitude markedly decreased in all animals and remained lowered after resuscitation with 100% oxygen at the time of peripheral collapse (as hemodynamic blood pressure and heart rate markers decrease). In animals that received treatment with quercetin nanosomes with 10 mg/kg of quercetin there was a significant recovery of brain electrical activity that was maintained up to 8 hours after the end of hypoxia (FIG. 7)

### Neuroprotective Effectiveness III

In the model of severe hypoxia in newborn pigs mentioned in the preceding paragraphs, following the experimental period of 8 hours, the animals were kept under intensive care for 72 hours, ensuring its survival. At 72 hours the electroencephalographic improvement seen at the end of experimental hypoxia persisted in animals treated with quercetin nanosomes. These animals also could suck a bottle and feed (80%) while only 20% of the animals subjected to hypoxia without treatment could. The latter required a greater oxygen supply assistance and inotropic and did not walk. Treated animals were able to walk, albeit with some difficulties.

e. Neuroprotective Effectiveness IV: Recovery of Dopamine Levels in the Striatum of Rats in Experimental Model of Parkinson's Disease.

In an experimental model of Parkinson's disease, the toxin 6-hydroxydopamine (6-OHDA) was injected into the Substance Nigra (SN) of adult male rats in a similar lesion to that observed in patients with Parkinson's disease. The neurotransmitter dopamine decreased in in the SN and striatum (ES), which is the area of the terminals of neurons of the SN. The administration of the nanosomal preparation in doses of 10 mg of quercetin 1 hour and 24 hours after the injury by 6-OHDA, significantly recovered the levels of dopamine in the ES, an index generally accepted as functional neurological recovery in the model (FIG. 5).

### REFERENCES

1. Reitsma, J B, et al, Epidemiology of stroke in The Netherlands from 1972 to 1994. The end of the decline in stroke mortality. Neuroepidemiology, 1998 17 (3):. P. 121-31.
2. Roger, V I. , Et al, Executive summary:. Heart disease and stroke statistics 2012 update˜: a report from the American Heart Association. Circulation, 2012 125 (1): p.. 188-97.
6. Ketzoian, C, Medical Uruguayan Press, 1997 18. P. 9-26.
7. Evans, D J, Levene M I, and M. Tsakmakis, Anticonvulsants for mortality and morbidity preventative in full term newborns with perinatal asphyxia. Cochrane Database Syst Rev, 2007 (3): p. CD001240.
9. Shankaran, S., et al., Acute neonatal morbidity and long-term Central nervous system sequelae of perinatal asphyxia in term infants. Early Hum Dev, 1991 25 (2):. P. 135-48.
10. The Naqeeb, N., et al., Assessment of neonatal encephalopathy by amplitude-integrated electroencephalography. Pediatrics, 1999 103 (6 Pt 1). P. 1263-71.
12. Vasiljevic, B., S. aglajlic-Djukic, and M. Gojnic, The prognostic Value of amplitude-integrated electroencephalography in neonates with hypoxic-ischemic encephalopathy. Vojnosanit Pregl, 2012 69 (6): p.. 492-9.
13. Du Plessis, A J and M V Johnston, Hypoxic-ischemic brain injury in the newborn. Cellular Mechanisms and potential strategies for neuroprotection. Clin Perinatol 1997 24 (3):. P. 627-54.
14. Palmer C, Vannucci R C, and J. Towfighi, Reduction of perinatal hypoxic-ischemic brain damage with allopurinol. Pediatr Res, 1990, 27 (4 Pt 1). P. 332-6.
15. Shankaran, S., et al, Childhood outcomes after neonatal encephalopathy for hypothermia, N Engl J Med 2012 366 (22):.. P. 2085-92.
16 Boots, A W, Haenen G R, and A. Bast, Health effects of quercetin: from antioxidant to nutraceutical. Eur J Pharmacol, 2008 585 (2-3). P. 325-37.
Dajas 17, F., Life or Death: neuroprotective and anticancer effects of quercetin. J Ethnopharmacol, 2012 143 (2): p.. 383-96.
18. Ossola, B., T M Kaariainen, and P T Mannisto, The multiple faces of quercetin in neuroprotection. Expert Opin Drug Saf, 2009 8 (4): p.. 397-409.
19. Kaariainen, T M, et al, Lack of robust protective effect of quercetin in two types of 6-. Hydroxydopamine-induced parkinsonian models in rats and cell cultures dopamlnerglc. Brain Res, 2008 1203. P. 149-59.
20. Dajas, F, et al. Neuroprotection by flavonoids. Braz J Med Biol Res, 2003, 36 (12). P.1613-20.
21 Dajas, F., et al., Cell culture protection and in vivo neuroprotective capacity of flavonoids. Neurotox Res, 2003 5 (6): p.. 425-32.
22. Rivera, F., et al., Reduction of ischemic brain damage and Increase of glutathione by a liposomal preparation of quercetin in permanent focal ischemia in rats. Neurotox Res,. 2008 13 (2): p. 105-14.
23. Arredondo, M F, et al., Cytopmtection by Achyrocline satureioides (Lam) DC and some of its main flavonoids against oxidative stress. J Ethnopharmacol, 2004, 91 (1): p. 13-20.
24. Lasic, D D and D. Papahadjopoulos, Liposomes revisited. Science, 1995 267 (5202). P.1275-6.
25. Mandal, A K and N. Das, Sugar coated liposomal flavonoid: a unique Formulation in Combating carbon tetrachloride induced hepatic oxidative damage. J Drug Target, 2005 13 (5): p. 305-15.
26. Goniotaki, M., et al, Encapsulation of naturally occurring flavonoids into liposomes. Physicoche ical properties and biological activity against human cancer cell Unes. J Pharm Pharmacol, 2004, 56 (10). P. 1217.
24. Rivera, F., et al, Some aspects of the in vivo neuroprotective capacity of flavonoids. Bioavailability and structure-activity relationship. Neurotox Res, 2004, 6 (7-8). P. 543-53. Yuan, ZP, et al., Liposomal quercetin Efficiently Suppresses growth of solid tumors in murine models. Clin Cancer Res, 2006, 12 (10). P. 3193-9.
25. Zhi-Pingyuan, et al Liposomal Quercetin Efficiently Suppresses Growth of Solid Tumors in Murine Models, Clin Cancer Res, 2006, 12 (10):.. P. 3193-3199.
26. Loftsson, T. and Brewster M E, Pharmaceutical applications of cyclodextrins. 1. Drug solubilization and stabilization. Sd J Pharm, 1996, 85 (10). P. 1017-25.
27. Ghosh, A., et al., Nanoencapsulation of dietary quercetin Enhances its efficacy in arsenic-induced oxidative Patriarchal damage in liver and brain of rats. Life Sci, 2009 84 (3-4): p.. 75-80.
28. Abdel-Rahman, S M, et al., Single-dose pharmacokinetics of intravenous itraconazole and hydroxypropyl-beta-cyclodextrin in infants, children, and adolescents. Antimicrob A

1. An injectable formulation for intravenous administration comprising nanosomes of lecithin/cholesterol containing the complex formed by flavone and flavonol or its alkyl and sulfur derivatives with 2-hydroxypropyl-cyclodextrin, dispersed in a physiological solution, wherein the proportion lecithin cholesterol is 1:5 to 1:10; the ratio of flavone and flavonol or alkylated and/or sulfur derivatives to 2-hydroxypropyl-p-cyclodextrin is in the form of 1:20 to 1:50; and the ratio of flavone and flavonol or alkylated or sulfur derivatives to cholesterol/lecithin is from 0.5:1 to 1:10.

2. An injectable formulation according to claim 1 wherein the flavone and flavonol or alkylated or sulfur derivatives is quercetin, wherein the ratio of cholesterol to lecithin is from 1:5 to 1:10; quercetin ratio to 2-hydroxypropyl-cyclodextrin is about 1:20 to 1:50 and the proportion of quercetin cholesterol/lecithin is from 0.5:1 to 1:2, dispersed in saline.

3. An injectable formulation for intravenous administration of nanosomes of lecithin/cholesterol complex comprising quercetin or other flavonol and flavone or its alkyl or sulfur derivatives with 2-hydroxypropyl-cyclodextrin, characterized in that it is prepared according to the following steps:
I—Preparation of the complex mixing 2-hydroxypropyl-3-cyclodextrin and quercetin or other flavonol or flavone on a ratio to 2-hydroxypropyl-β-cyclodextrin of 1:20 to 1:50 times for a period of 12 to 90 hours, in a medium containing ethanol with continuous stirring in sterile conditions.
II—Inclusion of the complex obtained in step I into a nanosome of lecithin/cholesterol in a ratio of 5 to 10 respectively, with sonication for a period of 5 min to 2 hours.
III—The preparation obtained in step II is mixed with the complex obtained in step I in a ratio of flavonoid/cholesterol of 0.5 to 2 times.
IV—The formulation obtained in III is injected into a saline solution at a temperature between 60 and 100 degrees Celsius at a rate of 5 to 30 ml/h to result in the formation of nanosomes.

4. An injectable formulation according to claim 3 wherein the flavonol used in steps I to III is quercetin.

5. Using the nanosomes containing the complex comprising quercetin or other flavonol and flavone and their alkylated or sulfur derivatives and /, 2-hydroxypropyl-cyclodextrin, obtained through the different steps of claim 3 for preparing a drug that acts as a neuroprotective agent for the treatment of acute episodes of stroke in adult and perinatal asphyxia in children.

6. Using the nanosomes containing the complex comprising quercetin or other flavonol and flavone and their alkylated or sulfur derivatives with 2-hydroxypropyl-cyclodextrin, obtained through the different steps of claim 3 for preparing a drug that acts as a neuroprotective agent in brain neurodegenerative processes.

7. Using the nanosomes containing the complex comprising quercetin or other flavonol and flavone and their alkylated or sulfur derivatives with 2-hydroxypropyl-cyclodextrin, obtained through the different steps of claim 3 for preparing a drug that acts as a neuroprotective agent for the skull-brain traumatic pathology.

8. A process whereby nanosomes obtained from the different steps of claim 3 are used in claim 5 by intravenous administration.

9. A process whereby nansomes obtained from the different steps of claim 3 are used in claim 6 by intravenous administration.

10. A process whereby nansomes obtained from the different steps of claim 3 are used in claim 7 by intravenous administration.
